(19) United States
(12) Patent Application Publication
(10) Pub. No.: US 2005/0191371 A1
(43) Pub. Date: Sep. 1, 2005

(54) Medical composition

(76) Inventor: Andres Marmolejo Rizo, Perris, CA (US)

Correspondence Address: MICHAEL A. SHIPPEY, PH. D., 4848 LAKEVIEW AVENUE, SUITE B, YORBA LINDA, CA, 92886, US

(21) Appl. No.: 10/777,359

(22) Filed: Feb. 12, 2004

(51) Int. Cl. A61K 31/19; A61K 35/78

(52) U.S. Cl. 424/717; 424/747; 514/557

(57) ABSTRACT

A novel composition for the treatment of skin and body infections, including rashes, blisters, sore throats, and fungal invasions, that is useful for topical application to the skin and throat and also for ingestion. Vinegar, baking soda, and wintergreen mint comprise its main active ingredients. It is a suitable alternative to prescription and over-the-counter remedies that are based on harmful substances such as steroids and phenols. Additional ingredients include mints, sweeteners, and flavorings, which together relieve the symptoms and eliminate the cause of skin and body infections.

### FIELD OF THE INVENTION

This invention relates generally to nonprescription, homeopathic remedies, and more particularly, to a homeopathic composition for application by spray or ingestion to effect healing of skin and body infections.

### DESCRIPTION OF RELATED ART

References to homeopathic medications are legion. Although homeopathic medications have only recently achieved a degree of widespread respect, such medications have been in use for eons, longer than the history of the written word. Some representative examples we have found are as follows:
1) U.S. Pat. No. 6,426,338 to T. J. Borody describes a colchicines/aminosalicylic acid blend for treatment of constipation.
2) U.S. Pat. No. 6,596,313 to R. A. Rosenbaum describes a cold remedy and swallowing aid.
3) U.S. Pat. No. 6,592,896 to R. A. Rosenbaum describes a cold remedy.

One of the most common and inexpensive medicine prescribed against bacterial infections is penicillin or any of a number of derivative antibiotics. This medicine has many disadvantages, including cost, length of time required to ingest it continuously to avoid recurrence of the infection, troublesome side effects, and a growing resistance against it by the very diseases it is supposed to eliminate. For a large percentage of the population, penicillin in any form causes severe allergic reactions, and therefore cannot be used. Such persons can be treated with a variety of other medicines that are more expensive, all of which have the same disadvantages noted above.

Effective treatments for a wide variety of illnesses caused by viruses and bacteria have long been sought, both in terms of symptom relief and antiviral and antimicrobal action. Medicines generally used to combat cold viruses treat the symptoms, and often contain ingredients shown to be harmful. For example, many products for treating sore throats contain phenol as an active ingredient. This ingredient is a simple alcohol/oxidative derivative of benzene. Both of these chemicals are listed as extremely carcinogenic by the Environmental Protection Agency.

Prior art medicinal applications for skin infections, including eczemas, rashes, blisters, athlete's foot, and fungal invasions have various disadvantages, including cost, use of steroids and hormonal substances like cortisone, difficulty in application, and limited effect requiring frequently reapplication. Non-steroidal medications are used to treat inflammations, as well. Such medications include acetaminophen, aspirin, ibuprofen, and naproxen. All of these medicinal applications have significant side effects, ranging from organ and tissue damage, particularly to stomach and kidney, to bone and bone marrow damage, to tissue swelling, cataracts, weight gain, and even high blood pressure. A major disadvantage to topical skin compositions is that the substances must be delivered directly to the point of the infection and must penetrate the skin adequately. This usually requires frequent application in a large quantity. It is common that such substances demonstrate significant signs of relief in laboratory conditions, but less than desired results because of the lack of an effective delivery system for reaching the tissue.

The medicinal compounds that have been developed in prior art have a further disadvantage of being comprised of a multitude of ingredients, rendering them complex and expensive to make and more susceptible to potential allergic reactions among a larger segment of the population.

There remains a need for a medicinal composition comprised of simple, natural, easily obtainable ingredients that is less expensive for the consumer and that can be used to cure a large variety of infections or diseases. Furthermore, there is a need for a medicinal composition that can be applied frequently during an infection without any significant side effects or allergic reactions. Said composition must meet the regulatory requirements of the Federal Drug Administration (FDA) and Homeopathic Pharmacopoeia of the United States (HPUS) as a homeopathic drug product.

### SUMMARY OF THE INVENTION

The present invention is a remedy for skin and body infections that contains a buffered solution of vinegar and baking soda, compounded with wintergreen, as its main active ingredient base in place of other substances known to have harmful and severe side effects. This active ingredient is combined with several other commonly used and ingestible ingredients to make the composition attractive to the consumer. The resulting formulation utilizes the little-known activities of a buffered solution of vinegar and baking soda, compounded with wintergreen, to reduce symptoms and eliminate the cause of skin and body infections. Gum acacia is also used in small amounts.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as representative basis for teaching one skilled in the art to employ the present invention in virtually any appropriately detailed system, structure or manner.

A Composition of the Present Invention

A composition of the present invention formulated in the form of a throat spray was prepared using the procedure described above. The ingredients of the throat spray are listed below:

TABLE 1
Preferred Potencies of Ingredients

| Ingredient/Source | Potency | Indication |
|---|---|---|
| 0.5 gallons distilled white vinegar | 5% (aq.) | |
| 30 g Na₂Co₃, baking soda | as delivered | |
| Wintergreen | 15 g | |
| Gum Acacia | 50 mg | |
| Flavoring and sweeteners: | total 30 g | |
| Sorbitol | | |
| Maltodextrin | | |
| Artificial flavors | | |
| Sweetener | | |
| Magnesium stearate | | |
| Gum acacia | | |
| Soy lecithin | | |
| Artificial color Blue 1 | | |
| Artificial color Yellow 5 | | |

The foregoing ingredients are provided in an aqueous base. Examples of natural sweeteners include, but are not limited to, fructose, sucrose, succinate, rice syrup, glucose, stevia, glycerin, honey, and barley malt. Examples of natural flavors include, but are not limited to, peppermint, spearmint, chamomile, etc.

The ingredients are blended together at room temperature, and stirred for 15 minutes. The resulting solution is used in a sprayer for a sore throat. It can also be applied topically to treat such conditions as rashes and blisters.

The level of wintergreen can be raised to increase potency. In the above example, the sum of the weight of wintergreen and the combined weight of all minor ingredients (those listed below wintergreen) will be held at 45 g. Thus, when the level of wintergreen is raised, the level of minor ingredients is according reduced.

Case Studies

No formal clinical studies have been carried out on this medicinal composition. However, the inventor has used the formula in Table 1 to cure his athlete's foot. He has offered the composition to family members. They have used the composition to successfully treat skin conditions, including blisters and rashes. They have also used the composition to successfully treat sore throat.

While the invention has been described in connection with a preferred embodiment or embodiments, it is not intended to limit the scope of the invention to the particular form set forth, but on the contrary, it is intended to cover such alternatives, modifications, and equivalents as may be included within the spirit and scope of the invention as defined by the appended claims.

1. A medicinal composition comprising:
a. A major amount of a dilute aqueous solution of a naturally occurring organic acid;
b. An amount of a group I metal carbonate sufficient to react with 5%-50% of said acid, thereby producing a buffered solution; and,
c. A significant amount of wintergreen mint;
and wherein all ingredients are present in the composition in amounts effective together to provide substantial relief of one or more symptoms selected from the group consisting of symptoms of: a common cold, sore throat, congestion, skin inflammation, skin rash, skin blisters, athlete's foot, mouth and throat blisters.

2. A composition as claimed in claim 1, wherein the organic acid comprises acetic acid.

3. A composition as claimed in claim 1, wherein the metal carbonate comprises sodium carbonate.

4. A composition as claimed in claim 1, wherein the organic acid solution comprises distilled white vinegar.

5. A composition as claimed in claim 1, wherein the metal carbonate comprises baking soda.

6. A composition as claimed in claim 1, further comprising flavoring agents.

7. A composition as claimed in claim 1, wherein said composition is formulated into a form selected from the group consisting of lozenges, tablets, syrup, salve, and pills.

8. A composition as claimed in claim 1, further comprising a pharmaceutically acceptable carrier.

9. A medicinal composition comprising:
40-120 ml of vinegar, strength 3-10%
5-100 g baking soda
5-50 g wintergreen mint
5-5000 mg gum acacia
0.5-5 g sweetener; and
0.5-5 g artificial flavorings, wherein the base is water, and the ingredients listed are present in the composition in amounts effective together to provide substantial relief of one or more symptoms associated with soar throat when said composition is applied by spray to the throat at least 4 times daily until no longer needed.
